(19) United States
(12) Patent Application Publication
(10) Pub. No.: US 2026/0207494 A1
(43) Pub. Date: Jul. 23, 2026

(54) STABLE LIQUID FORMULATIONS COMPRISING (6S)-5-METHYLTETRAHYDROFOLIC ACID OR SALTS THEREOF

(71) Applicant: Merck Patent GbmH, Darmstadt (DE)

(72) Inventors: Viola Groehn, Dachsen (CH); Jean-Pierre Knapp, Schaffhausen (CH); Miriam Wildt, Altdorf (CH); Daniel Ritter, Altdorf (CH); Rudolf Moser, Schaffhausen (CH)

(21) Appl. No.: 19/157,845

(22) PCT Filed: Feb. 21, 2024
(86) PCT No.: PCT/EP2024/054329

§ 371 Date: Aug. 19, 2025

(30) Foreign Application Priority Data
Feb. 24, 2023 (EP) 23158466.5

(51) Int. Cl. A61K 9/08; A61K 31/519; A61K 47/22; A61K 47/46

(52) U.S. Cl. CPC A61K 9/08; A61K 31/519; A61K 47/22; A61K 47/46

(57) ABSTRACT

The present invention relates to stable liquid formulations comprising (6S)-5-methyltetrahydro-folic acid or salts thereof, to a process for obtaining the same, and to the use of such stable liquid formulations as liquid vitamin preparations or as ingredient in liquid vitamin preparations.

The present invention relates to a stable liquid formulation comprising (6S)-5-methyltetrahydrofolic acid or salts thereof, to a process for preparing said liquid formulation, and to the use of said liquid formulation as a liquid vitamin preparation or as an ingredient in a liquid vitamin preparation.

As used herein, (6S)-5-methyltetrahydrofolic acid, also referred to as L-5-methyltetrahydrofolate, L-5-MTHF, (6S)-5-methyltetrahydofolate or (6S)-5-MTHF, refers to levomefolic acid in its naturally occurring isomeric form N-[4-({[(6S)-2-amino-5,6,7,8-tetrahydro-4-hydroxy-5-methylpteridine-6-yl]methyl}amino)benzoyl]-L-glutamic acid, which is the primary biologically active form of folate used at the cellular level for DNA reproduction, the cysteine cycle and the regulation of homocysteine. It is also the main form of tetrahydrofolates found in circulation and transported across membranes in tissues and across the blood-brain barrier. In the cell, (6S)-5-methyltetrahydrofolic acid is used in the methylation of homocysteine to form methionine and tetrahydrofolate (THF). THF is the immediate acceptor of one carbon unit for the synthesis of thymidine-DNA, purines (RNA and DNA) and methionine. The oxidized and non-methylated form, folic acid, is a synthetic form of folate, and must undergo enzymatic reduction by dihydrofolate reductase (DHFR) to become biologically active.

### BACKGROUND OF THE INVENTION

Folates play an essential role in the metabolic regulation of amino acids and nucleic acids, and in one-carbon metabolism. As a result, they perform an important function in the body, aiding in proper cell division, DNA health and gene expression. Since folate cannot be produced by the human body, it must be obtained from the diet. Adequate folate intake is vital for cell division and for proper organism development and plays an important role in promoting the nervous system and in preventing birth defects, vascular and cardiovascular diseases, and cancer. A lack of folate is recognized to be at the base of many dysfunctions and abnormalities in a part of the population and in specific phases of life, like in the childbearing age and in pregnancy.

Hence, folate supplementation is strongly recommended in populations and risk for deficiency due to specific conditions. Folic acid is the synthetic form of the vitamin B₉, usually incorporated into foods and supplements (see Heffernan et al., Nutrition & Metabolism, 2021, 18, 104, 1-11). However, in the body, synthetic folic acid must be reduced by cell metabolism into the bioactive (6S)-5-methyltetrahydrofolic acid, which is the bioavailable form of folate found in blood and necessary for the conversion of the amino acid homocysteine to essential methionine. The body uses methionine to synthesize proteins and other important compounds. However, a common polymorphism in the gene coding for methylenetetrahydrofolatereductase (MTHFR) is associated with a decreased activity of the enzyme. People with this genetic deficiency are impaired to convert folic acid provided via dietary supplements into the bioactive(6S)-5-methyltetrahydrofolic acid. MTHFR polymorphisms also have been linked to increased risks of several pathologies, including cancer. For those who have this polymorphism but are unaware of it, supplementing their diet with folic acid will be ineffective and eventually lead to critical unmetabolized folic acid circulating in the body.

In recent years, more and more research has been conducted on the physiological activity of L-5-MTHF and salts thereof for humans, in particular with the aim of circumventing MTHFR polymorphisms. Good prospects arise when L-5-MTHF or a salt thereof is used as a supplement to prevent folate deficiency in humans, especially during pregnancy (see N. Miraglia et al., Alternative Therapies, May/June 2022, 28, 4, 12-17).

Synthetic folic acid is stable under most conditions of food processing and storage. The reduced tetrahydrofolates, such as 5-formyltetrahydrofolic acid, 5-methyltetrahydrofolic acid (L-5-MTHF) or tetrahydrofolic acid, however, are susceptible to degradation. Various oxidative and non-oxidative degradation mechanisms of folates have been extensively studied and described in the literature (see Gazzali et al., Eur. J. Pharm. Sci., 2016, 93, 419-430; Fitzhugh, Pteridines, 1993, 4, 187-191). In this context, it should be noted that not only the sensitivities of different folates to oxygen differ from one another, but also that their oxidative degradation pathways differ fundamentally from one another. For example, in the case of folic acid, exposure to UV light is usually required first to initiate an oxidative degradation process. In addition, the pH value plays a crucial role for various folates which is particularly important when preparing samples for analytics (see De Brouwer et al., Phytochem. Anal., 2007, 18, 496-508; Jastrebova et al., Eur. Food Res. Technol., 2013, 237, 747-754). For example, to reduce losses of 5-formyl-tetrahydrofolic acid during sample preparation, the use of two antioxidants, namely ascorbic acid in combination with 2-mercaptoethanol or 2,3-dimercapto-1-propanol, and a pH close to neutral were found to be of advantage. Moreover, various antioxidants, such as e.g. cinnamic acids, flavonoids, catechols, stilbenes, p-benzoquinones, isoprenoids, curcumin, oleic acid and linoleic acid were investigated for their potential to prevent folic acid photodecomposition (see Wusigale et al., J. Agric. Food Chem. 2020, 68, 340-350).

While L-5-MTHF is reasonably stable in solid crystalline form, it degrades relatively quickly in amorphous state and especially in solution, thereby reducing the effective amount available. In a first step of an oxidative degradation, L-5-methyl-5,6-dihydrofolate, L-5-methyl-5,8-dihydrofolate, and 4α-hydroxy-5-methyl-5,6,7,8-tetrahydrofolate are formed (see Ratanasthien et al., J. Clin. Path., 1977, 30, 438-448; Blair et al., J. C. S. Perkin II, 1975, 18-21). Further reaction leads to an irreversible chemical rearrangement of the pteroic acid heterocycle (see Whiteley et al., Biochem. Biophys. Res. Comm., 1981, 101, 4, 1259-1265). The resulting pyrazino-s-triazine derivative (L-Mefox) lacks vitamin activity. In view of the susceptibility of the naturally occurring folates to degradation, it is not surprising that the folates content of foods may decrease substantially during cooking, or even during storage at ambient temperature (see Indrawati et al., J. Agric. Food Chem., 2004, 52, 486-492).

There is still a great need for improvement, particularly for mid-term and long-term stabilization of liquid formulations comprising L-5-MTHF and salts thereof, which may be used in (1) liquid vitamin preparations in the field of dietary supplements, such as dietary supplements for population groups with increased or unmet folate requirements, such as athletes (sports nutrition), females who are pregnant or lactating or want to become pregnant (nutrition during (pre)pregnancy and lactation), individuals who have difficulty swallowing solid dosage forms, such as children and the elderly, or individuals who want to improve their well-being (vitamin shots or vitamin drinks); (2) liquid vitamin preparations for fortification of foodstuff, such as cereals and dairy products (e.g. flour, yoghurt, etc.); (3) liquid vitamin preparations for life science applications, such as liquid vitamin preparations for stabilizing cell culture media; (4) pharmaceutical and/or cosmetic preparations, such as eye drops; and (5) non-pharmaceutical preparations for over-the-counter (OTC) items, such as plasters and wound dressings (wound healing). Here, special attention is directed to industrially manufactured liquid vitamin preparations, which may or may not be based on natural food sources. Liquid vitamin preparations can be used in the form of sprays, drops or drinking bottles.

In this context, however, it should be emphasized that successful stabilization of folates depends on many different factors, such as e.g. the chemical properties of the folate (e.g. reduced or non-reduced folate, substituted or unsubstituted folate, type of substitution, etc.), the chemical properties of the stabilizing agent, the properties of the formulation (e.g. solid, liquid, dispersion, emulsion, etc.), the presence of additives and/or cosolvents. Beyond that, further physical parameters, such as e.g. temperature, exposure to radiation (e.g. light), pressure and pH play an important role. As mentioned above, various different folate degradation pathways are known in the prior art, which differ greatly from one another, both from chemical and mechanistical point of view. Having this in mind, the skilled person is well aware that the results of a specific application cannot simply be transferred to another application in the field of folate stabilization. Each case must be looked at individually.

For example, a comparative study on pressure and temperature stability of 5-methyltetrahydrofolic acid in model systems and in food products showed that ascorbic acid increases the stability of 5-MTHF. The study shows that (i) degradation of 5-MTHF in model systems follows a first-order reaction kinetics, (ii) thermostability of 5-MTHF increases at higher pH up to 7, (iii) 5-MTHF is relatively pressure-stable at temperatures of <40° C., and (iv) ascorbic acid improves both thermos- and barostability of 5-MTHF (see Indrawati et al., J. Agric. Food Chem., 2004, 52, 486-492). However, this study does not address the industrial manufacturing of mid-term and long-term stabilized liquid formulations comprising L-5-MTHF and salts thereof as described in the present invention.

In a review from 2020, the stability and interaction of folates with biological molecules, such as antioxidants, carbohydrates, proteins and nucleic acid was discussed (see Wusigale et al., J. Agric. Food Res., 2020, 2, 100039, 1-7). It was found that folates degrade under light, heat, oxygen, and acidic pH and that antioxidants may allow a better preservation of folates. This is particularly the case with fruits and vegetables rich in antioxidants, such as ascorbic acid and polyphenols, which may allow a better preservation of folates. This effect has been verified in food systems for ascorbic acid and 5-MTHF. In this regard, it was found that ascorbic acid at 0.1-0.5 mg/g diminished the degradation of folates at 0.1-0.6 μg/g in food products (i.e. orange juice, kiwi puree, carrot juice and asparagus). However, this review does not address the industrial manufacturing of mid-term and long-term stabilized liquid formulations comprising L-5-MTHF and salts thereof as described in the present invention.

In addition, there are numerous publications describing the stabilization of liquid or solid formulations containing folates with natural antioxidants. However, these publications relate to either solid formulations with folic acid (see U.S. Pat. No. 8,747,915 B1 and US 2004/0001817 A1) or liquid formulations with folic acid (see US 2019/0060336 A1; US 2007/0190209 A1; US 2009/0061023 A1; WO 2015/099842 A1; and EP 0954986 A2). However, none of these publications specifically addresses L-5-MTHF, which differs fundamentally from folic acid in terms of stability and degradation mechanisms. In addition, none of these publications addresses mid-term and long-term stability of liquid formulations comprising L-5-MTHF. However, liquid L-5-MTHF formulations are useful for the above-mentioned fields of application (1), (2), (3), (4) and (5), and particularly for individuals who have difficulty swallowing solid dosage forms, such as, for example, children and the elderly.

Stability is a critical property and component of formulations containing compounds that can easily decompose under the influence of certain parameters. Chemical stability studies are performed both in solution and solid state. It is an established fact that solution state and solid-state stability can differ both qualitatively and quantitatively. The stability of a crystalline material and an amorphous material, such as a lyophilisate, can also differ in the solid state. Extensive studies are conducted on the chemical stability of an active ingredient and its formulations by exposure to a variety of stressors, such as high temperature and/or high humidity. These studies also provide valuable information about the degradation products and help in developing meaningful specifications and the intrinsic stability of formulations of the active ingredient. Most common pathways for active ingredient degradation include, inter alia hydrolysis, oxidation, and photochemical degradation.

### OBJECT OF THE INVENTION

The purpose of the present invention is to overcome the disadvantages and drawbacks in the prior art and to provide a liquid formulation containing (6S)-5-methyltetrahydrofolic acid or salts thereof, which (a) shows mid-term and/or long-term stability at ambient and elevated temperature, (b) contains generally recognized as safe (GRAS) substances, and (c) is particularly suitable for use in (1) liquid vitamin preparations in the field of dietary supplements, such as dietary supplements for population groups with increased or unmet folate requirements, such as athletes (sports nutrition), females who are pregnant or lactating or want to become pregnant (nutrition during (pre)pregnancy and lactation), individuals who have difficulty swallowing solid dosage forms, such as children and the elderly, or individuals who want to improve their well-being (vitamin shots or vitamin drinks); (2) liquid vitamin preparations for fortification of foodstuff, such as cereals and dairy products (e.g. flour, yoghurt, etc.); (3) liquid vitamin preparations for life science applications, such as liquid vitamin preparations for stabilizing cell culture media; (4) pharmaceutical and/or cosmetic preparations, such as eye drops; and (5) non-pharmaceutical preparations for over-the-counter (OTC) items, such as plasters and wound dressings (wound healing). Liquid formulations containing (6S)-5-methyltetrahydrofolic acid or salts thereof can be used in the form of sprays, drops or drinking bottles.

Hence, it is an object of the present invention to provide a liquid formulation containing (6S)-5-methyltetrahydrofolic acid or a salt thereof and showing the above-mentioned beneficial effects (a) to (c). It is a further object of the present invention to provide a liquid formulation containing (6S)-5-methyltetrahydrofolic acid or a salt thereof, which shows the above-mentioned beneficial effects (a) to (c) and which can be used as a liquid vitamin preparation or as an ingredient in a liquid vitamin preparation.

Finally, it is an object of the present invention to provide a process for preparing said liquid formulations.

The purpose of stability testing is to provide evidence on how the quality of a product varies with time under the influence of a variety of environmental factors, such as temperature, humidity, and light, and to establish a suitable shelf life for the active ingredient product and recommended storage conditions.

### SUMMARY OF THE INVENTION

It has now surprisingly been found that certain liquid formulations comprising (6S)-5-methyltetrahydrofolic acid or a salt thereof, one or more antioxidant(s) selected from grape seed extract, rosemary extract or components thereof, and a liquid formulation medium overcome the previously discussed known drawbacks and allow for the preparation of stable liquid formulations of (6S)-5-methyltetrahydrofolic acid or a salt thereof, which are particularly suitable for use in (1) liquid vitamin preparations in the field of dietary supplements, such as dietary supplements for population groups with increased or unmet folate requirements, such as athletes (sports nutrition), females who are pregnant or lactating or want to become pregnant (nutrition during (pre)pregnancy and lactation), individuals who have difficulty swallowing solid dosage forms, such as children and the elderly, or individuals who want to improve their well-being (vitamin shots or vitamin drinks); (2) liquid vitamin preparations for fortification of foodstuff, such as cereals and dairy products (e.g. flour, yoghurt, etc.); (3) liquid vitamin preparations for life science applications, such as liquid vitamin preparations for stabilizing cell culture media; (4) pharmaceutical and/or cosmetic preparations, such as eye drops; and (5) non-pharmaceutical preparations for over-the-counter (OTC) items, such as plasters and wound dressings (wound healing).

The liquid formulation according to the present invention shows mid-term and/or long-term stability at ambient and elevated temperature and contains ingredients which are substances generally recognized as safe (i.e. GRAS substances). The liquid formulation according to the present invention can be used in the form of sprays, drops or drinking bottles.

The addition of one or more antioxidant(s) selected from grape seed extract, rosemary extract or components thereof has surprisingly been found to provide mid-term and even long-term stabilization to liquid formulations containing (6S)-5-methyltetrahydrofolic acid or a salt thereof. In particular, the decrease in the content of reduced folate in the formulation due to conversion or degradation is significantly slowed down or even stopped. As a result, the content of the reduced folate used remains constant over a longer period of time, which allows the manufacturing of vitamin containing preparations with a content of L-5-MTHF that is stable over a certain period of time.

The liquid formulation obtained is stable over months, preferably up to 3 months, more preferably up to 6 months, even more preferably up to 12 months, even more preferably up to 18 months and most preferably up to 18 months and beyond, without significant loss of active ingredient, i.e. the amount of L-5-MTHF after the time specified is maintained at or above 40%, preferably at or above 45%, more preferably at or above 50%, even more preferably at or above 55%, even more preferably at or above 60%, even more preferably at or above 65%, even more preferably at or above 70%, even more preferably at or above 75%, even more preferably at or above 80%, even more preferably at or above 85%, even more preferably at or above 90%, and most preferably at or above 95%. This enables the storage and use of the liquid formulations according to the present invention for the manufacturing of formulations or preparations with L-5-MTHF without significant decomposition.

Hence, the present invention relates to the following embodiments:

A liquid formulation comprising:
(a) (6S)-5-methyltetrahydrofolic acid or a salt thereof;
(b) one or more antioxidant(s) selected from grape seed extract, rosemary extract or components thereof; and
(c) a liquid formulation medium.

A process for preparing a liquid formulation according to the present invention, wherein the process comprises the following steps (i) and (ii); or (i′) and (ii′):
(i) dissolving (6S)-5-methyltetrahydrofolic acid or a salt thereof in a liquid formulation medium; and
(ii) adding one or more antioxidant(s) selected from grape seed extract, rosemary extract or components thereof;
or
(i′) dissolving one or more antioxidant(s) selected from grape seed extract, rosemary extract or components thereof in a liquid formulation medium; and
(ii′) adding (6S)-5-methyltetrahydrofolic acid or a salt thereof.

Use of a liquid formulation according to the present invention as a liquid vitamin preparation or as an ingredient in a liquid vitamin preparation.

Preferred embodiments of the present invention are described hereinafter in the dependent claims.

### BRIEF DESCRIPTION OF THE FIGURES

FIG. 1: Long-term stability of L-5-MTHF in Example 1-2 (▪) and Reference Example 1-1 (●) at storage conditions of 25° C. and 60% relative humidity (RH). The content (%) of L-5-MTHF mono-sodium salt as determined by LC-MS/MS according to the method AOAC 2013.13 is plotted over time.

FIG. 2: Long-term stability of L-5-MTHF in Examples 1-1 (▴), Example 1-2 (▪) and Reference Example 1-1 (●) at storage conditions of 40° C. and 75% relative humidity (RH). The content (%) of L-5-MTHF mono-sodium salt as determined by LC-MS/MS according to the method AOAC 2013.13 is plotted over time.

### DETAILED DESCRIPTION

### Definitions

In the context of the present invention, the term “formulation medium” as used herein, denotes one or more compounds serving as a solvent, emulsion agent, suspending agent, carrier and/or matrix for the (6S)-5-methyltetrahydrofolic acid or salt thereof and any other component of the liquid formulation. A formulation medium typically consists of a compound or a mixture of several compounds that are inert vis-à-vis said (6S)-5-methyltetrahydrofolic acid or salt thereof and said other component(s) contained in the liquid formulation. In the context of the present invention, the formulation medium is a liquid and provides the basis for the liquid formulation. Exemplary formulation media are water or mixtures of water and ethanol.

In the context of the present invention, the term “antioxidant” as used herein, refers to a substance that inhibits oxidation, a chemical reaction that can produce free radicals. This can lead to degradation reactions of oxidation-sensitive compounds. Antioxidants are frequently added to industrial products, such as fuels and lubricants, to prevent oxidation, and to foods to prevent spoilage, particularly oils and fats to become rancid. Dietary antioxidants are vitamins A, C and E.

In the context of the present invention, the term “mid-term stabilization”, as used here, designates a stabilization that occurs over a period of 3 to 6 months.

In the context of the present application, the term “long-term stabilization”, as used here, designates a stabilization that occurs over a period of >6 months, preferably 9 months, more preferably 12 months. Preferred periods for long-term stabilization are from >6 to 24 months, >6 to 18 months, 9 to 18 months and 12 to 18 months.

In the context of the present invention, the term “ambient temperature” as used herein, refers to a temperature in the range from about 20 to about 25° C., preferably to a temperature of about 20° C.

Throughout this specification and the claims which follow, unless the context requires otherwise, the word “comprise”, and variations, such as “comprises” or “comprising”, will be understood to imply the inclusion of a stated compound, or class or group of compounds, or group of steps but not the exclusion of any other integer or step or group of integers or steps.

### PREFERRED EMBODIMENTS

### Liquid Formulation

The present invention relates to a liquid formulation comprising:
(a) (6S)-5-methyltetrahydrofolic acid or a salt thereof;
(b) one or more antioxidant(s) selected from grape seed extract, rosemary extract or components thereof; and
(c) a liquid formulation medium.

Preferably, the liquid formulation according to the present invention comprises one, two, three, four, five, six, seven, eight, nine or ten antioxidant(s) selected from grape seed extract, rosemary extract or components thereof. More preferably, the liquid formulation according to the present invention comprises one, two, three, four or five antioxidant(s) selected from grape seed extract, rosemary extract or components thereof. Even more preferably, the liquid formulation according to the present invention comprises one, two or three antioxidant(s) selected from grape seed extract, rosemary extract or components thereof. Most preferably, the liquid formulation according to the present invention comprises one antioxidant selected from grape seed extract, rosemary extract or components thereof.

In a preferred embodiment of the present invention, the antioxidant in the liquid formulation is selected from grape seed extract or components thereof. Grape seed extract is an industrial derivative of whole grape seeds and contains proanthocyanidins. Properties and ingredients of grape seed extract are described in Chen et al., Journal of Functional Foods, 73, 2020, 104113, 1-18. Here (poly)phenolic compounds, such as, for example, catechins, epicatechins, proanthocyanidins, kaempferol, quercetin, and gallic acid are described as antioxidants found in grape seed extract.

Preferably, the antioxidant selected from grape seed extract or components thereof is selected from the group consisting of catechin, epicatechin, epicatechin gallate, epigallocatechin gallate, gentisic acid, syringic acid, protocatechuic acid, gallic acid, caffeic acid, procyanidin B₁, procyanidin B₂, procyanidin B₃, procyanidin B₄, procyanidin B₁-3-O-gallate (B₁-G), procyanidin B₂-3-O-gallate (B₂-G), procyanidin B₂-3′-O-gallate (B₂-G′), procyanidin C₁, kaempferol, and quercetin. More preferably, the antioxidant selected from grape seed extract or components thereof is selected from the group consisting of catechin, and caffeic acid.

In an alternative preferred embodiment of the present invention, the antioxidant in the liquid formulation is selected from rosemary extract or components thereof. Rosemary extract is a botanical extract used as food additive. Properties and ingredients of rosemary extract are described in Xie et al., Eur. J. Lipid Sci. Technol., 2017, 119, 1600439, 1-10. Here (poly)phenolic compounds, such as, for example, carnosic acid, carnosol, and rosmarinic acid are described as antioxidants found in rosemary extract.

Preferably, the antioxidant selected from rosemary extract or components thereof is selected from the group consisting of carnosic acid, carnosol, and rosmarinic acid. More preferably, the antioxidant selected from rosemary extract or components thereof is selected from the group consisting of carnosic acid, and rosmarinic acid.

In a further alternative embodiment of the present invention, the antioxidant selected from grape seed extract, rosemary extract or components thereof is selected from the group consisting of catechin, epicatechin, epicatechin gallate, epigallocatechin gallate, gentisic acid, syringic acid, protocatechuic acid, gallic acid, caffeic acid, procyanidin B₁, procyanidin B₂, procyanidin B₃, procyanidin B₄, procyanidin B₁-3-O-gallate (B₁-G), procyanidin B₂-3-O-gallate (B₂-G), procyanidin B₂-3′-O-gallate (B₂-G′), procyanidin C₁, kaempferol, quercetin, carnosic acid, carnosol, and rosmarinic acid. Preferably, the antioxidant selected from grape seed extract, rosemary extract or components thereof is selected from the group consisting of catechin, caffeic acid, carnosic acid, and rosmarinic acid.

Preferably, in the embodiments of the liquid formulation according to the present invention, the (6S)-5-methyltetrahydrofolic acid or salt thereof is selected from the group consisting of (6S)-5-methyltetrahydrofolic acid, (6S)-5-methyltetrahydrofolic acid calcium salt (Metafolin®), (6S)-5-methyltetrahydrofolic acid monosodium salt (Arcofolin®), (6S)-5-methyltetrahydrofolic acid disodium salt, (6S)-5-methyltetrahydrofolic acid L-isoleucine ethyl ester salt, (6S)-5-methyltetrahydrofolic acid L-leucine ethyl ester salt, and (6S)-5-methyltetrahydrofolic acid L-valine ethyl ester salt.

More preferably, in the embodiments of the liquid formulation according to the present invention, the (6S)-5-methyltetrahydrofolic acid or salt thereof is (6S)-5-methyltetrahydrofolic acid monosodium salt (Arcofolin®).

It is preferred in the embodiments of the liquid formulation according to the present invention that the liquid formulation medium is water.

As an alternative, it is preferred in the embodiments of the liquid formulation according to the present invention that the liquid formulation medium is a mixture of water and ethanol. Preferably, the content of ethanol in said mixture is in the range from 0.01 to 10.0 vol.-%, more preferably from 0.01 to 5.0 vol.-%, even more preferably from 0.01 to 1.0 vol.-%, and most preferably from 0.01 to 0.5 vol.-%, based on the total volume of the formulation medium.

It is preferred in the embodiments of the liquid formulation according to the present invention that the concentration of (6S)-5-methyltetrahydrofolic acid or salt thereof, calculated as free acid form, is in the range from 0.0001 to 10.0 mg/mL, preferably from 0.001 to 6.00 mg/mL, and more preferably from 0.001 to 4.00 mg/mL, based on the liquid formulation.

Typical concentrations of (6S)-5-methyltetrahydrofolic acid or salt thereof, calculated as free acid form, in sprays or drops are in the range from 1.00 to 10.0 mg/mL, preferably 2.00 to 7.00 mg/mL, more preferably 3.00 to 5.00 mg/mL, and most preferably about 4.00 mg/mL. Typical concentrations of (6S)-5-methyltetrahydrofolic acid or salt thereof, calculated as free acid form, in vitamin shots are in the range from 0.001 to 1.00 mg/mL, preferably 0.01 to 0.50 mg/mL, more preferably 0.05 to 0.10 mg/mL, and most preferably about 0.08 mg/mL. Typical concentrations of (6S)-5-methyltetrahydrofolic acid or salt thereof, calculated as free acid form, in vitamin drinks are in the range from 0.0001 to 0.100 mg/mL, preferably 0.0005 to 0.010 mg/mL, more preferably 0.001 to 0.005 mg/mL, and most preferably about 0.0016 mg/mL.

It is preferred in the embodiments of the liquid formulation according to the present invention that the concentration of the antioxidant(s) is in the range from 0.0001 to 300 mg/mL, preferably from 0.001 to 150 mg/mL, and more preferably from 0.004 to 34.0 mg/mL, based on the liquid formulation.

It is preferred in the embodiments of the liquid formulation according to the present invention that the molar equivalent of the antioxidant(s) to the (6S)-5-methyltetrahydrofolic acid or salt thereof is in the range from 0.01 to 4500 equiv., preferably from 0.01 to 2250 equiv., and more preferably from 0.01 to 450 equiv.

It is preferred in the embodiments of the liquid formulation according to the present invention that said liquid formulation further comprises L-ascorbic acid or a salt thereof, preferably sodium L-ascorbate. Preferably, the concentration of L-ascorbic acid or salt thereof, calculated as free acid form, is in the range from 0.001 to 200 mg/mL, preferably from 0.01 to 100 mg/mL, and more preferably from 0.1 to 10 mg/mL, based on the liquid formulation.

Preferably, the molar equivalent of the L-ascorbic acid or salt thereof and the antioxidant(s) is in the range from 0.0001 to 2000 equiv., preferably from 0.001 to 1000 equiv., and more preferably from 0.01 to 100 equiv.

It is preferred in the embodiments of the liquid formulation according to the present invention that said liquid formulation further comprises tocopherol or a derivative thereof, preferably tocopherol or tocopherol polyethylene glycol succinate (tocofersolan), more preferably tocopherol polyethylene glycol succinate (tocofersolan). Preferably, the concentration of the tocopherol or derivative thereof is in the range from 0 to 200 mg/mL, preferably from 0.001 to 50.0 mg/mL, and more preferably from 0.01 to 10.0 mg/mL, based on the liquid formulation.

It is preferred in the embodiments of the liquid formulation according to the present invention that said liquid formulation further comprises one or more compound(s) selected from the group consisting of L-isoleucine ethyl ester, L-leucine ethyl ester and L-valine ethyl ester. Said compounds may improve the solubility properties of the individual components comprised in the liquid formulation. Moreover, said compounds are particularly suitable as additives for dietary supplements, preferably for dietary supplements for athletes.

It is preferred in the embodiments of the liquid formulation according to the present invention that the pH of said liquid formulation is in the range from 5.0 to 10.0, preferably from 5.5 to 9.0, more preferably from 6.0 to 8.0, and most preferably from 6.4 to 8.0. The liquid formulation according to the present invention may contain a buffer to stabilize a specific pH. Suitable buffers are citrate buffer, aspartate buffer, and monopotassium phosphate (KH₂PO₄) buffer.

Furthermore, the liquid formulation according to the present invention may contain minerals of the type and quantity found in mineral water. Non-limiting examples of such minerals include magnesium, calcium, sodium, potassium, bicarbonate, sulphate, silica, chloride, and nitrate.

It is preferred that the liquid formulation according to the present invention is provided in a dosage form for use as a spray, drops or drinking bottle. Said drinking bottle can be either a simple drinking bottle containing the liquid vitamin preparation as a vitamin shot or vitamin drink or a drinking bottle, which contains the liquid vitamin preparation as a vitamin shot or vitamin drink and additionally contains one or more solid preparation(s) (e.g. tablets) of further vitamins and/or minerals (in combination also referred to as micronutrients), wherein said solid preparations are separated from said liquid vitamin preparation. For example, said solid preparation(s) may be located in a separated compartment of the drinking bottle, such as, for example, the cap.

### Process

Further provided is a process for preparing a liquid formulation according to the present invention, wherein the process comprises the following steps (i) and (ii); or (i′) and (ii′):
(i) dissolving (6S)-5-methyltetrahydrofolic acid or a salt thereof in a liquid formulation medium; and
(ii) adding one or more antioxidant(s) selected from grape seed extract, rosemary extract or components thereof;
or
(i′) dissolving one or more antioxidant(s) selected from grape seed extract, rosemary extract or components thereof in a liquid formulation medium; and
(ii′) adding (6S)-5-methyltetrahydrofolic acid or a salt thereof.

In a preferred embodiment of the process for preparing a liquid formulation according to the present invention, the formulation medium is water.

In an alternative preferred embodiment of the process for preparing a liquid formulation according to the present invention, the formulation medium is a mixture of water and ethanol, wherein preferably the content of ethanol is in the range from 0.01 to 10.0 vol.-%, preferably from 0.01 to 5.0 vol.-%, more preferably from 0.01 to 1.0 vol.-%, and most preferably from 0.01 to 0.5 vol.-%, based on the total volume of the formulation medium.

In a preferred embodiment of the process for preparing a liquid formulation according to the present invention, the one or more antioxidant(s) are added as a solution in step (ii), wherein preferably the solution contains the same liquid formulation medium of step (i).

In a preferred embodiment of the process for preparing a liquid formulation according to the present invention, (6S)-5-methyltetrahydrofolic acid or the salt thereof is added as a solution in step (ii′), wherein preferably the solution contains the same liquid formulation medium of step (i′).

As further preferred, more preferred, even more preferred and most preferred features of the process for preparing a liquid formulation according to the present invention, the preferred, more preferred, even more preferred and most preferred features, respectively, as disclosed above for the formulation can be applied.

### Use

The present invention relates to the use of the liquid formulation according to the present invention as a liquid vitamin preparation or as an ingredient in a liquid vitamin preparation.

Preferably, said liquid vitamin preparation is a dietary supplement, preferably a dietary supplement for population groups with increased or unmet folate requirements, more preferably a dietary supplement for athletes, females who are pregnant or lactating or want to become pregnant, individuals who have difficulty swallowing solid dosage forms and/or individuals who want to improve their well-being.

Preferably, said dietary supplement is in a dosage form for use as a spray, drops or drinking bottle. Said drinking bottle can be either a simple drinking bottle containing the liquid vitamin preparation as a vitamin shot or vitamin drink or a drinking bottle, which contains the liquid vitamin preparation as a vitamin shot or vitamin drink and additionally contains one or more solid preparation(s) (e.g. tablets) of further vitamins and/or minerals (in combination also referred to as micronutrients), wherein said solid preparations are separated from said liquid vitamin preparation. For example, said solid preparation(s) may be located in a separated compartment of the drinking bottle, such as, for example, the cap.

Preferably, said liquid vitamin preparation is a liquid vitamin preparation for fortification of foodstuff, preferably cereals and dairy products.

Preferably, said liquid vitamin preparation is a liquid vitamin preparation for life science applications, preferably for stabilizing cell culture media.

Preferably, said liquid vitamin preparation is a liquid pharmaceutical and/or cosmetic preparation, preferably eye drops.

Preferably, said liquid vitamin preparation is a liquid non-pharmaceutical preparation for over-the-counter (OTC) items, preferably plasters and wound-dressings.

The present invention is further illustrated by the examples following hereinafter which shall in no way be construed as limiting. A skilled person will acknowledge that various modifications, additions and alternations may be made to the invention without departing from the spirit and scope of the invention as defined in the appended claims.

### EXAMPLES

### Analytical Methods

The content of L-5-MTHF in the prepared samples of the liquid formulation was determined by LC-MS/MS according to the method AOAC 2013.13.

### Preparation of Liquid Formulations

### Example 1-1

Preparation of liquid formulation containing L-5-MTHF monosodium salt (Arcofolin®), sodium L-ascorbate and grape seed extract (GSE) in mineral water (Evian®).

### Reagents and Equipment Used:

L-5-MTHF monosodium salt, bulk (Arcofolin®) (Batch: ESY0008-XX); mineral water (Evian®); 20 mL GC-HS vials; sodium L-ascorbate; grape seed extract (GSE) solution (2%, 46.2% ethanol, ad 100% deionized water) (Mibelle AG Biochemistry, CH-5033 Buchs); analytical balance with printer; pH meter; 500 mL volumetric flask; 100 mL volumetric flask; 50 mL volumetric flask; 500 mL graduated cylinder.

### Experimental Procedure:

1) Preparing a diluted grape seed extract (GSE) solution (1:100, 0.02%) with 0.5 mL grape seed extract (GSE) solution (2%) in 50 mL water. The concentration of grape seed extract (GSE) is 0.2 mg/mL. The solution can be stored in the refrigerator (dark and cool) and reused if necessary.
2) Preparing a Solution 1 with 35 mg L-5-MTHF monosodium salt (Arcofolin®) in 100 mL water. The concentration of L-5-MTHF monosodium salt is 0.3500 mg/mL.
3) Adding 3 mL of Solution 1, 500 mg sodium L-ascorbate and 10 mL of the diluted grape seed extract (GSE) solution (1:100, 0.02%) to a 500 mL volumetric flask and filling up to 500 mL with mineral water (Evian®). The concentration of L-5-MTHF monosodium salt is 0.0021 mg/mL, the concentration of sodium L-ascorbate is 1.0000 mg/mL and the concentration of grape seed extract (GSE) is 0.004 mg/mL. Step 3) should be performed immediately after step 2).
4) Measuring pH of the prepared solution.
5) Aliquoting the solution into 17×20 mL GC HS vials (20 mL each).
6) Sealing the GC-HS vials with crimping pliers.
7) Labeling: 2 vials: “Example 1-1, initial”; 9 vials: “Example 1-1, 25° C./60% RH”; 6 vials: “Example 1-1, 40° C./75% RH”.

### Example 1-2

Preparation of liquid formulation containing L-5-MTHF monosodium salt (Arcofolin®), sodium L-ascorbate and rosemary extract (RME) in mineral water (Evian®).

### Reagents and Equipment Used:

L-5-MTHF monosodium salt, bulk (Arcofolin®) (Batch: ESY0008-XX); mineral water (Evian®); 20 mL GC-HS vials; sodium L-ascorbate; rosemary extract (RME) (30% rosmarinic acid) (Gustav Parmentier GmbH, D-60320 Frankfurt am Main); analytical balance with printer; pH meter; 500 mL volumetric flask; 100 mL volumetric flask; 500 mL graduated cylinder.

### Experimental Procedure:

1) Preparing a Solution 1 with 35 mg L-5-MTHF monosodium salt (Arcofolin®) in 100 mL water. The concentration of L-5-MTHF monosodium salt is 0.3500 mg/mL.
2) Adding 3 mL of Solution 1, 500 mg sodium L-ascorbate and 1750 mg rosemary extract (RME) (30% rosmarinic acid) to a 500 mL volumetric flask and filling up to 500 mL with mineral water (Evian®). The concentration of L-5-MTHF monosodium salt is 0.0021 mg/mL, the concentration of sodium L-ascorbate is 1.0000 mg/mL and the concentration of rosemary extract (RME) (30% rosmarinic acid) is 3.5000 mg/mL. Step 2) should be performed immediately after step 1).
3) Measuring pH of the prepared solution.
4) Aliquoting the solution into 17×20 mL GC HS vials (20 mL each).
5) Sealing the GC-HS vials with crimping pliers.
6) Labeling: 2 vials: “Example 1-2, initial”; 9 vials: “Example 1-2, 25° C./60% RH”; 6 vials: “Example 1-2, 40° C./75% RH”.

### Reference Example 1-1

Preparation of liquid formulation containing L-5-MTHF monosodium salt (Arcofolin®) and sodium L-ascorbate in mineral water (Evian©).

### Reagents and Equipment Used:

L-5-MTHF monosodium salt, bulk (Arcofolin®) (Batch: ESY0008-XX); mineral water (Evian®); 20 mL GC-HS vials; sodium L-ascorbate; analytical balance with printer; pH meter; 500 mL volumetric flask; 100 mL volumetric flask; 500 mL graduated cylinder.

### Experimental Procedure:

1) Preparing a Solution 1 with 35 mg L-5-MTHF monosodium salt (Arcofolin®) in 100 mL water. The concentration of L-5-MTHF monosodium salt is 0.3500 mg/mL.
2) Adding 3 mL of Solution 1 and 500 mg sodium L-ascorbate to a 500 mL volumetric flask and filling up to 500 mL with mineral water (Evian®). The concentration of L-5-MTHF monosodium salt is 0.0021 mg/mL and the concentration of sodium L-ascorbate is 1.0000 mg/mL. Step 2) should be performed immediately after step 1).
3) Measuring pH of the prepared solution.
4) Aliquoting the solution into 17×20 mL GC HS vials (20 mL each).
5) Sealing the GC-HS vials with crimping pliers.
6) Labeling: 2 vials: “Reference Example 1-1, initial”; 9 vials: “Reference Example 1-1, 25° C./60% RH”; 6 vials: “Reference Example 1-1, 40° C./75% RH”.

TABLE 1
Examples 1-1 and 1-2 and Reference Example 1-1. Composition of liquid formulations.

| | c(mg/mL) | c(mg/mL) | c(mg/mL) | c(mg/mL) | | |
|---|---|---|---|---|---|---|
| | L-5-MTHF mono- | Sodium L- | Grape seed | Rosemary | Formulation | |
| Example | sodium salt | ascorbate | extract (GSE) | extract (RME) | medium | pH |
| Example 1-1 | 0.0021 | 1.0000 | 0.004 | — | Evian ® | 7.91 |
| Example 1-2 | 0.0021 | 1.0000 | — | 3.5000 | Evian ® | 6.42 |
| | | | | (1.0500 | | |
| | | | | rosmarinic acid) | | |
| Reference | 0.0021 | 1.0000 | — | — | Evian ® | 7.79 |
| Example 1-1 | | | | | | |

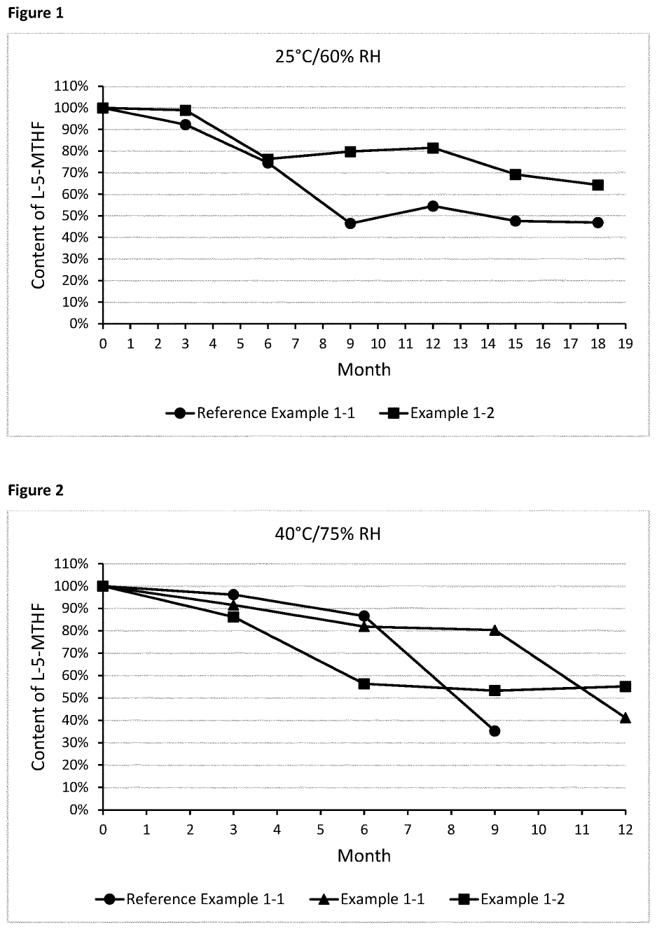

For long-term stability testing, 9 vials were stored in a climate chamber at 25° C. and 60% relative humidity (RH) and 6 vials were stored in a climate chamber at 40° C. and 75% relative humidity (RH). Samples were taken at periodic intervals and the content of L-5-MTHF mono-sodium salt was determined by HPLC according to the general procedure described above. The results are shown in FIGS. 1 and 2.

As can be seen from FIG. 1, Example 1-2 shows an unexpectedly improved long-term stability at 25° C. and 60% RH when compared to Reference Example 1-1.

As can be seen from FIG. 2, Examples 1-1 and 1-2 show an unexpectedly improved long-term stability at 40° C. and 75% RH when compared to Reference Example 1-1.

1. A liquid formulation comprising:
(a) (6S)-5-methyltetrahydrofolic acid or a salt thereof;
(b) grape seed extract or rosemary extract; and
(c) a liquid formulation medium.

2-6. (canceled)

7. The liquid formulation according to claim 1, wherein the (6S)-5-methyltetrahydrofolic acid or salt thereof is selected from the group consisting of (6S)-5-methyltetrahydrofolic acid, (6S)-5-methyltetrahydrofolic acid calcium salt, (6S)-5-methyltetrahydrofolic acid monosodium salt, (6S)-5-methyltetrahydrofolic acid L-isoleucine ethyl ester salt, (6S)-5-methyltetrahydrofolic acid L-leucine ethyl ester salt, and (6S)-5-methyltetrahydrofolic acid L-valine ethyl ester salt.

8. The liquid formulation according to claim 1, wherein the (6S)-5-methyltetrahydrofolic acid or salt thereof is (6S)-5-methyltetrahydrofolic acid monosodium salt.

9. The liquid formulation according to claim 1, wherein the liquid formulation medium is water, or a mixture of water and ethanol wherein the content of ethanol is in the range from 0.01 to 10.0 vol.-%, based on total volume of the formulation medium.

10. The liquid formulation according to claim 1, wherein the concentration of (6S)-5-methyltetrahydrofolic acid or salt thereof, calculated as free acid form, is in the range from 0.0001 to 10.0 mg/mL, based on the liquid formulation.

11. The liquid formulation according to claim 1, wherein the concentration of grape seed extract or rosemary extract is in the range from 0.0001 to 300 mg/mL, based on the liquid formulation.

12. The liquid formulation according to claim 1, wherein the molar equivalent of grape seed extract or rosemary extract to the (6S)-5-methyltetrahydrofolic acid or salt thereof is in the range from 0.01 to 4500 equiv.

13. The liquid formulation according to claim 1, wherein the liquid formulation further comprises L-ascorbic acid or a salt thereof.

14. The liquid formulation according to claim 1, wherein the liquid formulation further comprises L-ascorbic acid or a salt thereof and the concentration of L-ascorbic acid or salt thereof, calculated as free acid form, is in the range from 0.001 to 200 mg/mL, based on the liquid formulation.

15. The liquid formulation according to claim 1, wherein the liquid formulation further comprises L-ascorbic acid or a salt thereof and the molar equivalent of the L-ascorbic acid or salt thereof and grape seed extract or rosemary extract is in the range from 0.0001 to 2000 equiv.

16. The liquid formulation according to claim 1, wherein the liquid formulation further comprises tocopherol or a derivative thereof.

17. The liquid formulation according to claim 1, wherein the liquid formulation further comprises one or more compound(s) selected from the group consisting of L-isoleucine ethyl ester, L-leucine ethyl ester and L-valine ethyl ester.

18. The liquid formulation according to claim 1, wherein the pH of the liquid formulation is in the range from 5.0 to 10.0.

19. The liquid formulation according to claim 1, wherein the liquid formulation is in a dosage form for use as a spray, drops or drinking bottle.

20. A process for preparing a liquid formulation according to claim 1, wherein the process comprises the following steps (i) and (ii); or (i′) and (ii′):
(i) dissolving (6S)-5-methyltetrahydrofolic acid or a salt thereof in a liquid formulation medium; and
(ii) adding grape seed extract or rosemary extract;
or
(i′) dissolving grape seed extract or rosemary extract in a liquid formulation medium; and
(ii′) adding (6S)-5-methyltetrahydrofolic acid or a salt thereof.

21. The process for preparing a liquid formulation according claim 20, wherein the formulation medium is water, or a mixture of water and ethanol wherein the content of ethanol is in the range from 0.01 to 10.0 vol.-%, based on total volume of the formulation medium.

22-28. (canceled)

29. The liquid formulation according to claim 1, wherein the pH of the liquid formulation is in the range from 6.0 to 8.0.

30. The liquid formulation according to claim 1, wherein the pH of the liquid formulation is in the range from 6.4 to 8.0.

31. The liquid formulation according to claim 1, wherein the concentration of (6S)-5-methyltetrahydrofolic acid or salt thereof, calculated as free acid form, is in the range from 0.001 to 4.00 mg/mL, based on the liquid formulation.

32. The liquid formulation according to claim 1, wherein the concentration of grape seed extract or rosemary extract is in the range from 0.004 to 34.0 mg/mL, based on the liquid formulation.
